(19) United States
(12) Patent Application Publication
(10) Pub. No.: US 2023/0068767 A1
(43) Pub. Date: Mar. 2, 2023

(54) DYNAMIC SEATING ADJUSTMENT BASED ON ERGONOMIC TWIN SIMULATION

(71) Applicant: International Business Machines Corporation, Armonk, NY (US)

(72) Inventors: Sarbajit K. Rakshit, Kolkata (IN); Mukundan Sundararajan, Bangalore (IN)

(21) Appl. No.: 17/411,130

(22) Filed: Aug. 25, 2021

(51) Int. Cl. G16H 50/20; G16H 50/50; G16H 10/60

(52) U.S. Cl. CPC G16H 50/20; G16H 50/50; G16H 10/60

(57) ABSTRACT

In an approach to dynamic seating adjustment, a computer monitors a multi-seating arrangement. A computer identifies one or more users, each in a seat of the multi-seating arrangement. A computer retrieves a user profile associated with each of the one or more users. A computer receives data from one or more sensors. A computer creates an ergonomic digital twin model for each of the one or more users based on the associated user profile and the received sensor data. Based on the ergonomic digital twin model, a computer creates a simulation of an experience of each of the one or more users. Based on the simulation, a computer predicts discomfort associated with sitting in the seat of the multi-seating arrangement is expected for one or more of the one or more users. A computer adjusts a configuration of the respective seat of the one or more users.

### BACKGROUND OF THE INVENTION

The present invention relates generally to the field of digital twins, and more particularly to dynamic seating adjustment based on ergonomic twin simulation.

A digital twin refers to a digital representation of a physical asset. In Internet of Things (IoT) systems, a digital twin can represent an evolving virtual data model that mimics the physical asset as well as the experiences and state changes of the physical asset. A digital twin may be said to store and track information about its twin physical asset. A digital twin marketplace allows manufacturers and suppliers to share digital resources associated with physical assets with owners and operators of the manufacturers' physical assets. Examples of digital resources include, but are not limited to, a bill of materials, warranty bulletins, warranty claims, maintenance plans, maintenance history, part replacement history, part usage history, specifications, 3-dimensional (3D) model and drawing data, operating manuals, usage data, operating history, ownership history, applicable standards, etc.

A digital twin for a human may have many uses in the field of healthcare. Once a digital twin is created, its use in healthcare is to be the test subject for treatments of diseases or injuries before use on the individual or persons at large. With enough biological data, more precise and effective medical interventions that are tailored to the individual patient can be realized. Because digital twins are digital models, they can be analyzed to provide the patient with ways to prevent future illness, provide direction for preventive maintenance, and even offer performance enhancements. The digital twin can act as a guide to health and personal maintenance to avoid and prevent future illness. A digital twin of a patient or organs allows surgeons and health professionals to practice procedures in a simulated environment rather than on a real patient. Sensors the size of bandages can monitor patients and produce digital models that can be monitored by artificial intelligence (AI) and used to improve care.

Ergonomics (or human factors) is the scientific discipline concerned with the understanding of interactions among humans and other elements of a system, and the profession that applies theory, principles, data and methods to design to optimize human well-being and overall system performance. Proper ergonomic design is necessary to prevent repetitive strain injuries and other musculoskeletal disorders, which can develop over time and can lead to long-term disability. Human factors and ergonomics are concerned with the “fit” between the user, equipment, and environment. It accounts for the user's capabilities and limitations in seeking to ensure that tasks, functions, information, and the environment suit that user.

### SUMMARY

Embodiments of the present invention disclose a computer-implemented method, a computer program product, and a system for dynamic seating adjustment based on ergonomic twin simulation. The computer-implemented method may include one or more computer processors monitoring a multi-seating arrangement. One or more computer processors identify one or more users, each in a seat of the multi-seating arrangement. One or more computer processors retrieve a user profile associated with each of the one or more users. One or more computer processors receive data from one or more sensors located throughout the multi-seating arrangement. One or more computer processors create an ergonomic digital twin model for each of the one or more users based on the associated user profile and the received sensor data. Based on the ergonomic digital twin model, one or more computer processors create a simulation of an experience of each of the one or more users, each in the respective seat of the multi-seating arrangement. Based on the simulation, one or more computer processors predict discomfort associated with sitting in the seat of the multi-seating arrangement is expected for one or more of the one or more users. One or more computer processors determine whether a seat adjustment is available for the respective seat of the one or more users. In response to determining the seat adjustment is available, one or more computer processors adjust a configuration of the respective seat of the one or more users.

### BRIEF DESCRIPTION OF THE DRAWINGS

FIG. 1 is a functional block diagram illustrating a distributed data processing environment, in accordance with an embodiment of the present invention;

FIG. 2 is a flowchart depicting operational steps of an ergonomic twin computing system, on a server computer within the distributed data processing environment of FIG. 1, for dynamic seating adjustment based on ergonomic twin simulation, in accordance with an embodiment of the present invention; and

FIG. 3 depicts a block diagram of components of the server computer executing the ergonomic twin computing system within the distributed data processing environment of FIG. 1, in accordance with an embodiment of the present invention.

### DETAILED DESCRIPTION

It is well known that prolonged sitting can have a plurality of adverse effects on the human body. Prolonged sitting may occur as a result of or working at a desk all day, long-distance travel, attendance at theater events, etc. Examples of issues resulting from prolonged sitting include weakened muscles, weight gain, back and neck pain, cancer, heart disease, diabetes, varicose veins, and deep vein thrombosis (DVT). During an event such as a long-distance flight or attendance at a long concert, play, or sporting event, a person's comfort can be one of the critical success factors to keeping the person satisfied with the experience. In period of prolonged sitting, a person can have various ergonomic problems. For example, the available space may not be sufficient to allow the person to stand up or stretch their legs. The person may not be able to sit properly in the available space, leading to possible health issues.

Embodiments of the present invention recognize that health and comfort issues may be prevented or minimized by providing a system that creates an ergonomic digital twin of individuals in a prolonged sitting situation to simulate and predict potential discomfort levels and take appropriate actions. Embodiments of the present invention can proactively adjust a seating configuration to improve comfort. Implementation of embodiments of the invention may take a variety of forms, and exemplary implementation details are discussed subsequently with reference to the Figures.

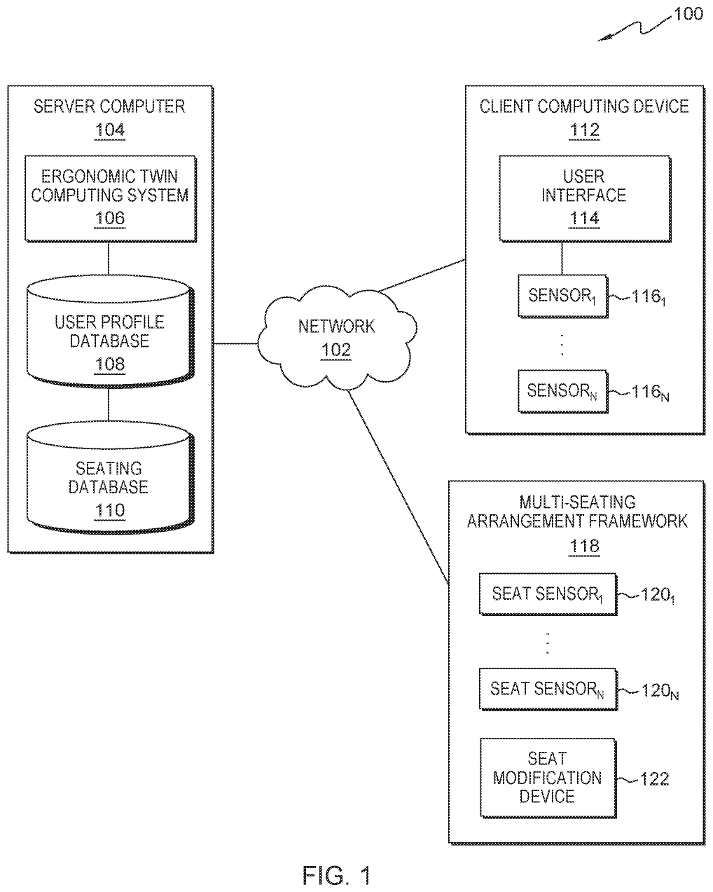

FIG. 1 is a functional block diagram illustrating a distributed data processing environment, generally designated 100, in accordance with one embodiment of the present invention. The term “distributed” as used herein describes a computer system that includes multiple, physically distinct devices that operate together as a single computer system. FIG. 1 provides only an illustration of one implementation and does not imply any limitations with regard to the environments in which different embodiments may be implemented. Many modifications to the depicted environment may be made by those skilled in the art without departing from the scope of the invention as recited by the claims.

Distributed data processing environment 100 includes server computer 104, client computing device 112, and multi-seating arrangement framework 118 interconnected over network 102. Network 102 can be, for example, a telecommunications network, a local area network (LAN), a wide area network (WAN), such as the Internet, or a combination of the three, and can include wired, wireless, or fiber optic connections. Network 102 can include one or more wired and/or wireless networks capable of receiving and transmitting data, voice, and/or video signals, including multimedia signals that include voice, data, and video information. In general, network 102 can be any combination of connections and protocols that will support communications between server computer 104, client computing device 112, multi-seating arrangement framework 118, and other computing devices (not shown) within distributed data processing environment 100.

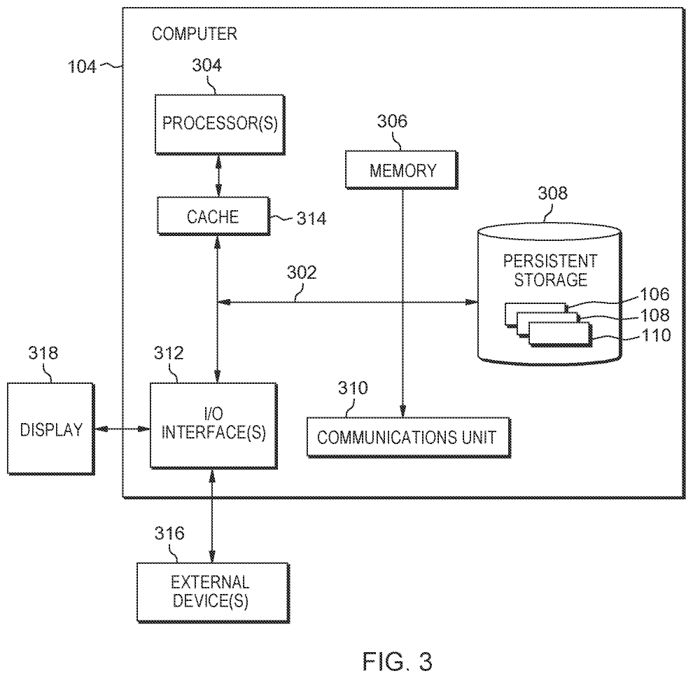

Server computer 104 can be a standalone computing device, a management server, a web server, a mobile computing device, or any other electronic device or computing system capable of receiving, sending, and processing data. In other embodiments, server computer 104 can represent a server computing system utilizing multiple computers as a server system, such as in a cloud computing environment. In another embodiment, server computer 104 can be a laptop computer, a tablet computer, a netbook computer, a personal computer (PC), a desktop computer, a personal digital assistant (PDA), a smart phone, or any programmable electronic device capable of communicating with client computing device 112 and other computing devices (not shown) within distributed data processing environment 100 via network 102. In another embodiment, server computer 104 represents a computing system utilizing clustered computers and components (e.g., database server computers, application server computers, etc.) that act as a single pool of seamless resources when accessed within distributed data processing environment 100. Server computer 104 includes ergonomic twin computing system 106, user profile database 108, and seating database 110. Server computer 104 may include internal and external hardware components, as depicted and described in further detail with respect to FIG. 3.

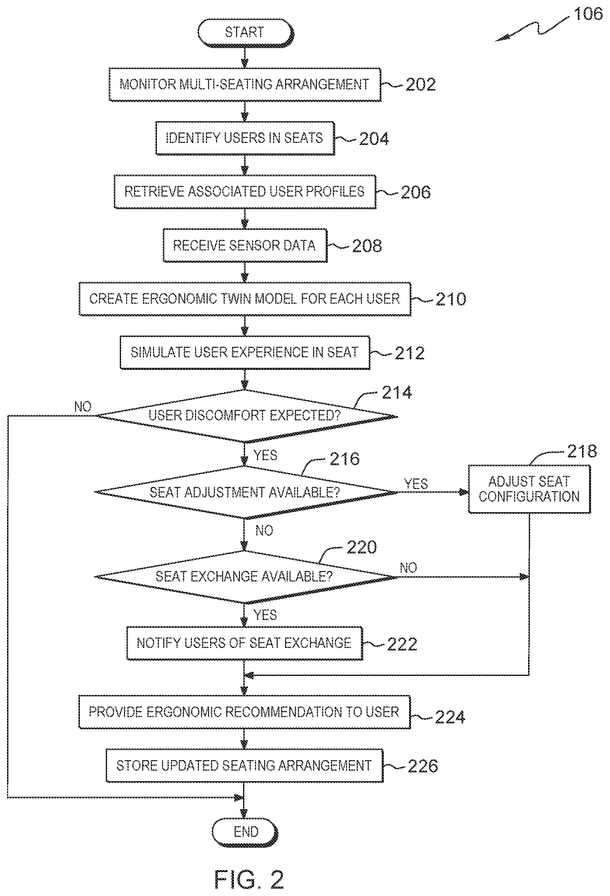

Ergonomic twin computing system 106 creates an ergonomic digital twin, herein referred to as an ergonomic twin, for each multi-seating arrangement or venue and for each user in the venue, and predicts, based on received data, whether a user is expected to experience discomfort in the seating arrangement in the duration of the event. Ergonomic twin computing system 106 monitors a multi-seating arrangement platform. Ergonomic twin computing system 106 identifies the users in the seats and retrieves associated user profiles. Ergonomic twin computing system 106 creates an ergonomic twin model for each user based on the retrieved profiles. Ergonomic twin computing system 106 simulates the seating experience for each user and determines whether user discomfort in the seat is expected. If discomfort is expected, then ergonomic twin computing system 106 determines if a seat adjustment is available and, if so, adjusts the seat configuration. If a seat adjustment is not available, ergonomic twin computing system 106 determines if a seat exchange between users is available and, if so, notifies the user of the seat exchange. Ergonomic twin computing system 106 provides an ergonomic recommendation to the users. Ergonomic twin computing system 106 stores the updated seating arrangement. Ergonomic twin computing system 106 is depicted and described in further detail with respect to FIG. 2.

User profile database 108 and seating database 110 each stores information used and generated by ergonomic twin computing system 106. In the depicted embodiment, user profile database 108 and seating database 110 reside on server computer 104. In another embodiment, user profile database 108 and seating database 110 may reside elsewhere within distributed data processing environment 100, provided that ergonomic twin computing system 106 has access to user profile database 108 and seating database 110. For example, user profile database 108 may reside on client computing device 112. A database is an organized collection of data. User profile database 108 and seating database 110 can each be implemented with any type of storage device capable of storing data and configuration files that can be accessed and utilized by ergonomic twin computing system 106, such as a database server, a hard disk drive, or a flash memory. User profile database 108 represents one or more databases that store a user profile for the user of client computing device 112. The user profile may include, but is not limited to, the name of the user, an address, an email address, a voice sample, a phone number, a credit card number, an account number, an employer, a job role, a job family, a job level, a resume, a medical record, a social network affiliation, etc. The user profile may also include user preferences for seating arrangements, such as seat size, seat spacing, seat angle, etc. User profile database 108 may also store data generated by sensor_{1-N} on client computing device 112. Seating database 110 stores seating arrangements of one or more venues associated with multi-seating arrangement framework 118. Seating database 110 may also store attributes and configurations of each of the seats included in a venue seating arrangement. For example, attributes and configurations may include, but are not limited to, a seat height range, a seat width range, a seat back angle range, a space between two seats—either front to back or side to side, a range of space between two seats, etc. Seating database 110 may also store historical data associated with previous venue seating arrangements. Seating database 110 may also store a digital twin model of the seating arrangement in the venue. Seating database 110 may also store medical information from one or more sources that describes indications and/or results of prolonged sitting.

The present invention may contain various accessible data sources, such as user profile database 108 and seating database 110, that may include personal data, content, or information the user wishes not to be processed. Personal data includes personally identifying information or sensitive personal information as well as user information, such as tracking or geolocation information. Processing refers to any operation, automated or unautomated, or set of operations such as collecting, recording, organizing, structuring, storing, adapting, altering, retrieving, consulting, using, disclosing by transmission, dissemination, or otherwise making available, combining, restricting, erasing, or destroying personal data. Ergonomic twin computing system 106 enables the authorized and secure processing of personal data. Ergonomic twin computing system 106 provides informed consent, with notice of the collection of personal data, allowing the user to opt in or opt out of processing personal data. Consent can take several forms. Opt-in consent can impose on the user to take an affirmative action before personal data is processed. Alternatively, opt-out consent can impose on the user to take an affirmative action to prevent the processing of personal data before personal data is processed. Ergonomic twin computing system 106 provides information regarding personal data and the nature (e.g., type, scope, purpose, duration, etc.) of the processing. Ergonomic twin computing system 106 provides the user with copies of stored personal data. Ergonomic twin computing system 106 allows the correction or completion of incorrect or incomplete personal data. Ergonomic twin computing system 106 allows the immediate deletion of personal data.

Client computing device 112 can be one or more of a laptop computer, a tablet computer, a smart phone, smart watch, a smart speaker, or any programmable electronic device capable of communicating with various components and devices within distributed data processing environment 100, via network 102. Client computing device 112 may be a wearable computer. Wearable computers are miniature electronic devices that may be worn by the bearer under, with, or on top of clothing, as well as in or connected to glasses, hats, or other accessories. Wearable computers are especially useful for applications that require more complex computational support than merely hardware coded logics. In one embodiment, the wearable computer may be in the form of a head mounted display. The head mounted display may take the form-factor of a pair of glasses. In an embodiment, the wearable computer may be in the form of a smart watch. In an embodiment, client computing device 112 may be integrated into a vehicle of the user. For example, client computing device 112 may include a heads-up display in the windshield of the vehicle. In general, client computing device 112 represents one or more programmable electronic devices or combination of programmable electronic devices capable of executing machine readable program instructions and communicating with other computing devices within distributed data processing environment 100 via a network, such as network 102. The user of client computing device 112 may be, for example, a passenger on a train or airplane, a viewer of a concert or sporting event, or a worker in a work environment that includes a multi-seating arrangement. Client computing device 112 includes an instance of user interface 114 and sensor 116_{1-N}.

User interface 114 provides an interface between ergonomic twin computing system 106 on server computer 104 and a user of client computing device 112. In one embodiment, user interface 114 is mobile application software. Mobile application software, or an “app,” is a computer program designed to run on smart phones, tablet computers and other mobile devices. In one embodiment, user interface 114 may be a graphical user interface (GUI) or a web user interface (WUI) and can display text, documents, web browser windows, user options, application interfaces, and instructions for operation, and include the information (such as graphic, text, and sound) that a program presents to a user and the control sequences the user employs to control the program. User interface 114 enables a user of client computing device 112 to input a user profile and/or preferences to be stored in user profile database 108.

User interface 114 also enables a user of client computing device 112 to receive recommendations from ergonomic twin computing system 106. In addition, user interface 114 enables a user of client computing device 112 to provide feedback to ergonomic twin computing system 106 regarding comfort in the seating arrangement and any actions taken by ergonomic twin computing system 106 to improve the user experience.

Multi-seating arrangement framework 118 is a system that is operatively coupled with the seats in the multi-seating arrangement and communicates with ergonomic twin computing system 106 regarding current seating arrangements and data generated by sensor 120_{1-N}. Multi-seating arrangement framework 118 includes seat sensor 120_{1-N} and seat modification device 122.

A sensor is a device that detects, collects, and/or measures a physical property and then records or otherwise responds to that property, such as vibration, chemicals, radio frequencies, environment, weather, humidity, light, etc. Sensor 116_{1-N} and sensor 120_{1-N}, herein sensor(s) 116 and sensor(s) 120, detect a plurality of attributes of client computing device 112 and multi-seating arrangement framework 118, respectively. As used herein, N represents a positive integer, and accordingly the number of scenarios implemented in a given embodiment of the present invention is not limited to those depicted in FIG. 1. Sensor(s) 116 and sensor(s) 120 may be one or more of a plurality of types of camera, including, but not limited to, pinhole, stereo, omni-directional, non-central, infrared, video, digital, three dimensional, panoramic, filter-based, wide-field, narrow-field, telescopic, microscopic, etc. In some embodiments, sensor(s) 116 and sensor(s) 120 include any device capable of imaging a portion of the electromagnetic spectrum. Sensor(s) 116 and sensor(s) 120 may be one or more of a plurality of types of microphone for detecting speech and other audible sounds. Sensor(s) 116 and sensor(s) 120 may be able to detect weather conditions, such as air temperature, relative humidity, presence and type of precipitation, wind speed, etc. Sensor(s) 116 and sensor(s) 120 may be GPS sensors. For example, sensor(s) 116 may use GPS, beacons, and/or Bluetooth® sensors that can detect geo-location or movement of the user of client computing device 112. Sensor(s) 116 and sensor(s) 120 may also be one or more of a plurality of types of near-field communications sensors. Sensor(s) 116 and sensor(s) 120 may be able to detect Wi-Fi® signals.

Sensor(s) 116 may be one or more biometric sensors for detecting the physical condition of the user, such as blood pressure, heart rate, respiratory rate, calories burned, calories consumed, pulse, oxygen levels, blood oxygen level, glucose level, blood pH level, salinity of user perspiration, skin temperature, galvanic skin response, electrocardiography data, body temperature, eye tracking data, etc. Sensor(s) 116 may also detect user movement, mobility, and activity patterns. Sensor(s) 120 may include various types of motion sensors, pressure sensors and/or strain gauges to detect a user in a seat, a user standing in an aisle of the venue, a change in position of a seat, etc. Sensor(s) 120 may include the ability to detect space occupied by a user. For example, a child may occupy less space than an adult. In another example, a passenger carrying luggage may occupy more space than a passenger that is not carrying anything. In one embodiment, sensor(s) 116 and sensor(s) 120 transmit data directly to seating database 110.

Seat modification device 122 is an apparatus operatively coupled with the seats in a multi-seating arrangement that can mechanically change the arrangement based on instructions generated by ergonomic twin computing system 106. For example, seat modification device 122 may be a hydraulic slider in the floor of the venue that can change the position of one or more seats such that ergonomic twin computing system 106 can change the spacing between the seats. In another example, seat modification device 122 may be operatively coupled with each seat in the venue such that ergonomic twin computing system 106 can change the configuration of an individual seat, e.g., the seat height or the seat back angle.

FIG. 2 is a flowchart depicting operational steps of ergonomic twin computing system 106, on server computer 104 within distributed data processing environment 100 of FIG. 1, for dynamic seating adjustment based on ergonomic twin simulation, in accordance with an embodiment of the present invention.

Ergonomic twin computing system 106 monitors multi-seating arrangement framework 118 (step 202). In an embodiment, ergonomic twin computing system 106 continually monitors multi-seating arrangement framework 118 to determine whether one or more users are in the seats of the venue with which multi-seating arrangement framework 118 is associated. In another embodiment, multi-seating arrangement framework 118 triggers ergonomic twin computing system 106 to begin monitoring once a first user occupies a seat. In a further embodiment, multi-seating arrangement framework 118 triggers ergonomic twin computing system 106 to begin monitoring once one or more of sensor(s) 120 begin communicating data. In an embodiment, ergonomic twin computing system 106 creates an ergonomic twin model of the seating arrangement in advance of an event and stores the model in seating database 110. The ergonomic twin model of the seating arrangement includes details, configurations, and specifications of the seats in the venue.

Ergonomic twin computing system 106 identifies the users in the seats (step 204). In an embodiment, as each user takes a seat in the venue, ergonomic twin computing system 106 identifies the user of the associated seat. In an embodiment, ergonomic twin computing system 106 identifies a user based on video or static imaging of the venue. In another embodiment, ergonomic twin computing system 106 identifies a user based on a reservation or ticket associated with the seat. In yet another embodiment, ergonomic twin computing system 106 identifies a user when the user, i.e., the user of client computing device 112, signals arrival in the seat via user interface 114. In a further embodiment, ergonomic twin computing system 106 identifies a user by associating client computing device 112 with a user account. For example, client computing device 112 may be a smart phone associated with an airline frequent flyer account.

Ergonomic twin computing system 106 retrieves associated user profiles (step 206). In an embodiment, ergonomic twin computing system 106 retrieves a user profile associated with each of the users in the seats of the venue from user profile database 108. As described above, the user profile may include preferences for seating arrangements, for example, a desired amount of space between the user and the seat in front of the user. The user profile may also include medical conditions, for example, details of back or knee problems. The user profile may also include physical characteristics of the user, such as height and weight. In general, the user profile may include one or more attributes which can influence the user's comfort in a prolonged sitting situation.

Ergonomic twin computing system 106 receives sensor data (step 208). In an embodiment, ergonomic twin computing system 106 receives data collected by sensor(s) 120 from multi-seating arrangement framework 118. In another embodiment, ergonomic twin computing system 106 receives data collected by sensor(s) 120 directly. In yet another embodiment, sensor(s) 120 transmit data to seating database 110, and ergonomic twin computing system 106 retrieves the sensor data from seating database 110. Sensor(s) 120 are associated with the seats in multi-seating arrangement framework 118 and can measure any ergonomic adjustments that a user makes to a seat once the user occupies the seat. For example, sensor(s) 120 may be associated with a seat on an airplane and detect the angle of the seat back. In another example, sensor(s) 120 may detect the weight of the person that occupies the seat. In an embodiment, ergonomic twin computing system 106 receives data generated by sensor(s) 116 associated with client computing device 112. In another embodiment, sensor(s) 116 transmit data to user profile database 108, and ergonomic twin computing system 106 retrieves the sensor data from user profile database 108. Sensor(s) 116 may collect biometric feedback from the user of client computing device 112, which can indicate comfort or discomfort while sitting in a seat of the venue. For example, sensor(s) 116 can detect an increase in blood pressure and/or heart rate, which may indicate that the user is experiencing pain. In an embodiment, ergonomic twin computing system 106 creates ergonomic measurements based on the sensor data received after the one or more users have settled into their seats. For example, ergonomic twin computing system 106 may determine a space delta, e.g., the amount of leg space or the amount of space between seats, based on the received sensor data. In an embodiment, ergonomic twin computing system 106 receives data from sensor(s) 116 from which ergonomic twin computing system 106 can determine a user's movement, mobility, and/or activity patterns.

Ergonomic twin computing system 106 creates an ergonomic twin model for each user (step 210). In an embodiment, based on the retrieved user profiles and/or the received sensor data, ergonomic twin computing system 106 creates an ergonomic twin model for each user in the multi-seating arrangement. In an embodiment, ergonomic twin computing system 106 may also use historical data associated with each specific user in the creation of the ergonomic twin model. For example, ergonomic twin computing system 106 may use data associated with the last time the user was in the venue with the multi-seating arrangement, e.g., in an entertainment venue or on a plane. In an embodiment, ergonomic twin computing system 106 may also use historical data associated with a plurality of other users that previously occupied that seat in the venue in the past. For example, ergonomic twin computing system 106 may use data associated with how often users that occupied a specific seat stood up over the course of an hour.

Ergonomic twin computing system 106 simulates the user experience for each seat (step 212). In an embodiment, ergonomic twin computing system 106 runs the ergonomic twin model for each user in the venue in order to simulate the sitting experience of the users over the duration of the event for which the user resides in that seat. The simulation predicts any discomfort the user may experience and predicts the timing of the discomfort as it relates to the duration of the event. The simulation may also predict the recovery time the user needs to be relieved of the discomfort. In an embodiment, ergonomic twin computing system 106 applies one or more medical sources related to prolonged sitting, stored in seating database 110, to the ergonomic twin model to simulate the sitting experience of the user. For example, ergonomic twin computing system 106 may retrieve one or more medical journals with articles related to the effects of prolonged sitting. In an embodiment, in addition to the medical sources, ergonomic twin computing system 106 may apply historical data associated with the seating arrangement, stored in seating database 110, to the ergonomic twin model. For example, ergonomic twin computing system 106 may retrieve historical images that depict users sitting in a slumped position in the seat. In an embodiment, ergonomic twin computing system 106 runs the ergonomic twin model of the user in conjunction with the digital twin model of the seating arrangement.

Ergonomic twin computing system 106 determines whether user discomfort in the seat is expected (decision block 214). In an embodiment, based on the ergonomic twin model simulation, ergonomic twin computing system 106 determines whether one or more of the users in the venue will experience discomfort from the seating arrangement, the seat position, and/or the duration of time spent in a seated position over the course of the event.

If discomfort is expected (“yes” branch, decision block 214), then ergonomic twin computing system 106 determines if a seat adjustment is available (decision block 216). In an embodiment, ergonomic twin computing system 106 retrieves data associated with the seat in the venue from seating database 110 to determine whether an adjustment can be made to the seat of the user predicted to be in discomfort that will minimize the discomfort of the user. In an embodiment, ergonomic twin computing system 106 retrieves data associated with the capabilities of seat modification device 122 to determine whether an appropriate seat adjustment is available. For example, if the user is predicted to have back pain after one hour in the seat, then ergonomic twin computing system 106 determines if seat modification device 122 has the capability to change the angle of the seat back.

If a seat adjustment is available (“yes” branch, decision block 216), then ergonomic twin computing system 106 adjusts the seat configuration (step 218). In an embodiment, ergonomic twin computing system 106 transmits instructions to seat modification device 122 to dynamically adjust the seat of the user to prevent or minimize discomfort. For example, ergonomic twin computing system 106 may transmit instructions for seat modification device 122 to increase the height of the seat by one inch to improve the angle of the legs of the user. In an embodiment where seat adjustment controls are present on the seat and available for the user to adjust, ergonomic twin computing system 106 transmits a notification to the user, via user interface 114, with instructions for a seat adjustment. For example, ergonomic twin computing system 106 may transmit a notification that states, “To prevent leg cramps halfway through the event, use the button on the right arm rest to slide the seat back two inches.” In an embodiment, prior to adjusting the seat configuration, ergonomic twin computing system 106 determines whether a seating adjustment for a first user may cause discomfort for a second user, and, if so, ergonomic twin computing system 106 may determine a different seat adjustment for the first user and/or the second user. For example, if the venue is an airplane, ergonomic twin computing system 106 may determine that reclining the seat back 100 percent for the first user will cause the user behind the first user to be uncomfortable, and therefore ergonomic twin computing system 106 reclines the seat back 50 percent.

If a seat adjustment is not available (“no” branch, decision block 216), then ergonomic twin computing system 106 determines if a seat exchange between users is available (decision block 220). In an embodiment, ergonomic twin computing system 106 determines whether two or more users may benefit from sharing, or exchanging, a particular seat over the duration of the event. For example, on a two-hour flight, a first user is seated on an aisle, such that the first user can stand up or stretch their legs periodically, while a second user is seated in a center seat of the row. Ergonomic twin computing system 106 determines that if the two users exchange seats after one hour, then neither user will experience discomfort during the flight. In an embodiment, ergonomic twin computing system 106 is trained with how ergonomic measurements and seat exchanges across multiple instances have benefitted users and been successful in order to predict a successful seat exchange and create a seat exchange portfolio. In an embodiment, ergonomic twin computing system 106 may determine one or more seat exchanges within a zone of the venue, such as a row of a theater or airplane, or across regions of the venue, such as the balcony of a theater. In an embodiment, ergonomic twin computing system 106 ranks the predicted discomfort of all the users in the venue to determine which user's discomfort is most critical to prevent or minimize and determines a seat exchange priority based on the ranking.

In an embodiment, ergonomic twin computing system 106 determines a recovery time associated with either the seat adjustment or the seat exchange. For example, the simulation may indicate that the user will need to stretch their legs after one hour but will feel better after stretching their legs for five minutes. Therefore, ergonomic twin computing system 106 determines the seat adjustment can revert back to the original configuration after five minutes.

If ergonomic twin computing system 106 determines a seat exchange is available (“yes” branch, decision block 220), then ergonomic twin computing system 106 notifies the users of the seat exchange (step 222). In an embodiment, ergonomic twin computing system 106 notifies the two or more users designated for a seat exchange via user interface 114. For example, ergonomic twin computing system 106 may transmit a notification to the two users that states, “To prevent discomfort, one hour from now, please exchange seats with the passenger in seat 9A/12B.” In an embodiment, the user of client computing device 112 includes a willingness to exchange seats in the user profile stored in user profile database 108.

Ergonomic twin computing system 106 provides an ergonomic recommendation to the user (step 224). In an embodiment, responsive to adjusting the seat configuration or notifying users of a seat exchange, or if neither of those options is available (“no” branch decision block 216 or “no” branch, decision block 220), ergonomic twin computing system 106 provides a recommendation to the user to improve or prevent any ergonomic discomfort predicted for a user. In an embodiment, ergonomic twin computing system 106 provides the recommendation via user interface 114. For example, ergonomic twin computing system 106 may transmit a notification that states, “After one hour, stand up and walk around for ten minutes.” In another example, ergonomic twin computing system 106 may transmit a notification that states, “Every half hour, extend your legs out straight.”

Ergonomic twin computing system 106 stores the updated seating arrangement (step 226). In an embodiment, ergonomic twin computing system 106 stores the seating arrangement that includes seat adjustments and/or seat exchanges in seating database 110. By storing updated seating arrangements that are based on the ergonomic twin models of the users, as well as the digital twin model of the venue and/or medical information, ergonomic twin computing system 106 creates a knowledge corpus that improves the predictions of ergonomic twin computing system 106 in the future. In an embodiment, ergonomic twin computing system 106 receives feedback from one or more of the users, via user interface 114, regarding whether the actions taken by ergonomic twin computing system 106 were beneficial. In the embodiment, ergonomic twin computing system 106 stores the feedback in seating database 110 to improve the knowledge corpus.

In an embodiment where a user is booking a seat for a future event in a multi-seating arrangement venue, ergonomic twin computing system 106 can determine a recommended seat for the user. In an embodiment, ergonomic twin computing system 106 determines the recommendation based on historical ergonomic data for users in the venue. Historical data may include image analysis of past users in the multi-seating arrangement, which can indicate whether any of the users displayed discomfort in their seats. In another embodiment, ergonomic twin computing system 106 determines the recommendation based on the user profile associated with the user, which includes preferences and health and/or medical information. In yet another embodiment, ergonomic twin computing system 106 determines the recommendation after receiving medical information associated with the user from a member institution of a medical network.

FIG. 3 depicts a block diagram of components of server computer 104 within distributed data processing environment 100 of FIG. 1, in accordance with an embodiment of the present invention. It should be appreciated that FIG. 3 provides only an illustration of one implementation and does not imply any limitations with regard to the environments in which different embodiments can be implemented. Many modifications to the depicted environment can be made.

Server computer 104 can include processor(s) 304, cache 314, memory 306, persistent storage 308, communications unit 310, input/output (I/O) interface(s) 312 and communications fabric 302. Communications fabric 302 provides communications between cache 314, memory 306, persistent storage 308, communications unit 310, and input/output (I/O) interface(s) 312. Communications fabric 302 can be implemented with any architecture designed for passing data and/or control information between processors (such as microprocessors, communications and network processors, etc.), system memory, peripheral devices, and any other hardware components within a system. For example, communications fabric 302 can be implemented with one or more buses.

Memory 306 and persistent storage 308 are computer readable storage media. In this embodiment, memory 306 includes random access memory (RAM). In general, memory 306 can include any suitable volatile or non-volatile computer readable storage media. Cache 314 is a fast memory that enhances the performance of processor(s) 304 by holding recently accessed data, and data near recently accessed data, from memory 306.

Program instructions and data used to practice embodiments of the present invention, e.g., ergonomic twin computing system 106, user profile database 108, and seating database 110, are stored in persistent storage 308 for execution and/or access by one or more of the respective processor(s) 304 of server computer 104 via cache 314. In this embodiment, persistent storage 308 includes a magnetic hard disk drive. Alternatively, or in addition to a magnetic hard disk drive, persistent storage 308 can include a solid-state hard drive, a semiconductor storage device, a read-only memory (ROM), an erasable programmable read-only memory (EPROM), a flash memory, or any other computer readable storage media that is capable of storing program instructions or digital information.

The media used by persistent storage 308 may also be removable. For example, a removable hard drive may be used for persistent storage 308. Other examples include optical and magnetic disks, thumb drives, and smart cards that are inserted into a drive for transfer onto another computer readable storage medium that is also part of persistent storage 308.

Communications unit 310, in these examples, provides for communications with other data processing systems or devices, including resources of client computing device 112 and multi-seating arrangement framework 118. In these examples, communications unit 310 includes one or more network interface cards. Communications unit 310 may provide communications through the use of either or both physical and wireless communications links. Ergonomic twin computing system 106, user profile database 108, seating database 110, and other programs and data used for implementation of the present invention, may be downloaded to persistent storage 308 of server computer 104 through communications unit 310.

I/O interface(s) 312 allows for input and output of data with other devices that may be connected to server computer 104. For example, I/O interface(s) 312 may provide a connection to external device(s) 316 such as a keyboard, a keypad, a touch screen, a microphone, a digital camera, and/or some other suitable input device. External device(s) 316 can also include portable computer readable storage media such as, for example, thumb drives, portable optical or magnetic disks, and memory cards. Software and data used to practice embodiments of the present invention, e.g., ergonomic twin computing system 106, user profile database 108, and seating database 110 on server computer 104, can be stored on such portable computer readable storage media and can be loaded onto persistent storage 308 via I/O interface(s) 312. I/O interface(s) 312 also connect to a display 318.

Display 318 provides a mechanism to display data to a user and may be, for example, a computer monitor. Display 318 can also function as a touch screen, such as a display of a tablet computer.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product. The computer program product may include a computer readable storage medium (or media) having computer readable program instructions thereon for causing a processor to carry out aspects of the present invention.

The computer readable storage medium can be any tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, a special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, a segment, or a portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The foregoing descriptions of the various embodiments of the present invention have been presented for purposes of illustration and example, but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer-implemented method comprising:
monitoring, by one or more computer processors, a multi-seating arrangement;
identifying, by one or more computer processors, one or more users, each in a respective seat of the multi-seating arrangement;
retrieving, by one or more computer processors, a user profile associated with each of the one or more users;
receiving, by one or more computer processors, data from one or more sensors located throughout the multi-seating arrangement;
creating, by one or more computer processors, an ergonomic digital twin model for each of the one or more users based on the associated user profile and the received sensor data;
based on the ergonomic digital twin model, creating, by one or more computer processors, a simulation of an experience of each of the one or more users, each in the respective seat of the multi-seating arrangement;
based on the simulation, predicting, by one or more computer processors, discomfort associated with sitting in the seat of the multi-seating arrangement is expected for one or more of the one or more users;
determining, by one or more computer processors, whether a seat adjustment is available for the respective seat of the one or more users; and
responsive to determining the seat adjustment is available, adjusting, by one or more computer processors, a configuration of the respective seat of the one or more users.

2. The computer-implemented method of claim 1, further comprising:
responsive to determining the seat adjustment is not available, determining, by one or more computer processors, a seat exchange is available between at least two users of the one or more users; and
notifying, by one or more computer processors, the at least two users of the seat exchange.

3. The computer-implemented method of claim 1, further comprising:
providing, by one or more computer processors, an ergonomic recommendation to prevent ergonomic discomfort to at least one of the one or more users.

4. The computer-implemented method of claim 3, wherein the ergonomic recommendation includes at least one of standing up, walking around, and extending legs.

5. The computer-implemented method of claim 1, wherein the configuration of the seat includes at least one of: a seat height range, a seat width range, a seat back angle range, a front to back space between two seats, a side to side space between two seats, and a range of space between two seats.

6. The computer-implemented method of claim 1, wherein the ergonomic digital twin model is based on at least one of: historical data associated with each of the one or more users and historical data associated with a plurality of other users that previously occupied the seat.

7. The computer-implemented method of claim 1, wherein the received sensor data is associated with one or more seats in the multi-seating arrangement.

8. The computer-implemented method of claim 1, further comprising:
storing, by one or more computer processors, an updated multi-seating arrangement.

9. A computer program product comprising:
one or more computer readable storage media and program instructions collectively stored on the one or more computer readable storage media, the stored program instructions comprising:
program instructions to monitor a multi-seating arrangement;
program instructions to identify one or more users, each in a respective seat of the multi-seating arrangement;
program instructions to retrieve a user profile associated with each of the one or more users;
program instructions to receive data from one or more sensors located throughout the multi-seating arrangement;
program instructions to create an ergonomic digital twin model for each of the one or more users based on the associated user profile and the received sensor data;
based on the ergonomic digital twin model, program instructions to create a simulation of an experience of each of the one or more users, each in the respective seat of the multi-seating arrangement;
based on the simulation, program instructions to predict discomfort associated with sitting in the seat of the multi-seating arrangement is expected for one or more of the one or more users;
program instructions to determine whether a seat adjustment is available for the respective seat of the one or more users; and
responsive to determining the seat adjustment is available, program instructions to adjust a configuration of the respective seat of the one or more users.

10. The computer program product of claim 9, the stored program instructions further comprising:
responsive to determining the seat adjustment is not available, program instructions to determine a seat exchange is available between at least two users of the one or more users; and
program instructions to notify the at least two users of the seat exchange.

11. The computer program product of claim 9, the stored program instructions further comprising:
program instructions to provide an ergonomic recommendation to prevent ergonomic discomfort to at least one of the one or more users.

12. The computer program product of claim 11, wherein the ergonomic recommendation includes at least one of standing up, walking around, and extending legs.

13. The computer program product of claim 9, wherein the configuration of the seat includes at least one of: a seat height range, a seat width range, a seat back angle range, a front to back space between two seats, a side to side space between two seats, and a range of space between two seats.

14. The computer program product of claim 9, wherein the ergonomic digital twin model is based on at least one of: historical data associated with each of the one or more users and historical data associated with a plurality of other users that previously occupied the seat.

15. A computer system comprising:
one or more computer processors;
one or more computer readable storage media;
program instructions collectively stored on the one or more computer readable storage media for execution by at least one of the one or more computer processors, the stored program instructions comprising:
program instructions to monitor a multi-seating arrangement;
program instructions to identify one or more users, each in a respective seat of the multi-seating arrangement;
program instructions to retrieve a user profile associated with each of the one or more users;
program instructions to receive data from one or more sensors located throughout the multi-seating arrangement;
program instructions to create an ergonomic digital twin model for each of the one or more users based on the associated user profile and the received sensor data;
based on the ergonomic digital twin model, program instructions to create a simulation of an experience of each of the one or more users, each in the respective seat of the multi-seating arrangement;
based on the simulation, program instructions to predict discomfort associated with sitting in the seat of the multi-seating arrangement is expected for one or more of the one or more users;
program instructions to determine whether a seat adjustment is available for the respective seat of the one or more users; and
responsive to determining the seat adjustment is available, program instructions to adjust a configuration of the respective seat of the one or more users.

16. The computer system of claim 15, the stored program instructions further comprising:
responsive to determining the seat adjustment is not available, program instructions to determine a seat exchange is available between at least two users of the one or more users; and
program instructions to notify the at least two users of the seat exchange.

17. The computer system of claim 15, the stored program instructions further comprising:
program instructions to provide an ergonomic recommendation to prevent ergonomic discomfort to at least one of the one or more users.

18. The computer system of claim 17, wherein the ergonomic recommendation includes at least one of standing up, walking around, and extending legs.

19. The computer system of claim 15, wherein the configuration of the seat includes at least one of: a seat height range, a seat width range, a seat back angle range, a front to back space between two seats, a side to side space between two seats, and a range of space between two seats.

20. The computer system of claim 15, wherein the ergonomic digital twin model is based on at least one of: historical data associated with each of the one or more users and historical data associated with a plurality of other users that previously occupied the seat.
